(19) United States
(12) Patent Application Publication
(10) Pub. No.: US 2026/0177515 A1
(43) Pub. Date: Jun. 25, 2026

(54) MULTICHANNEL INTEGRATED SYSTEMS AND METHODS FOR MULTIPARAMETER SENSING

(71) Applicant: GE Infrastructure Technology LLC, Greenville, SC (US)

(72) Inventors: Radislav Alexandrovich Potyrailo, Niskayuna, NY (US); Jeremy Daniel Popp, Niskayuna, NY (US)

(21) Appl. No.: 18/999,969

(22) Filed: Dec. 23, 2024

(51) Int. Cl. G01N 27/02; A61B 5/053; A61B 5/145

(52) U.S. Cl. CPC G01N 27/02; A61B 5/053; A61B 5/14546

(57) ABSTRACT

An impedance analyzer chip assembly may include a plurality of sensing regions, where each region of the plurality of sensing regions is configured to sense a gas, chemical, physical, or biological analyte. The impedance analyzer chip assembly may also include one or more processors configured to output one or more re-configurable impedance values across a frequency range of at least six orders of magnitude, output one or more re-configurable impedance values from one or more of the sensing regions of the plurality of sensing regions, concurrently output two or more impedance values from two or more of the sensing regions of the plurality of sensing regions, and concurrently output one or more impedance values at two or more frequencies.

### GOVERNMENT LICENSE RIGHTS

This invention was made with government support under contract 1U01AA029324-01 awarded by the National Institute of Health. The government has certain rights in the invention.

### FIELD

One or more embodiments are disclosed that relate to an application specific integrated circuit as an impedance analyzer for detection of gas, chemical, physical, and biological species.

### BACKGROUND

Detection of gas, chemical, physical, and biological species in different complex environments such as liquids or gases with needed sensitivity and accuracy is needed in diverse applications such as medical diagnostics, environmental surveillance, industrial safety, process monitoring, homeland protection, and many others. Existing sensors provide continuous monitoring capability, but their poor accuracy limits their adoption into new applications in wearable, distributed, and unattended monitoring formats. Such limitations originate from sensor designs that have only a single output per sensor.

Thus, when detection accuracy and sensitivity are essential, instruments based on traditional analytical technologies that have multiple outputs (e.g. liquid and gas chromatography, mass spectrometry, ion mobility spectrometry) are preferred despite their limitations such as the need for periodic sampling, intermittent measurements, relatively high-power consumption, large size and others. These instruments based on traditional analytical technologies are often inconvenient and costly to operate but are an unavoidable alternative to existing sensors.

### BRIEF DESCRIPTION

In one or more embodiments, a sensor system includes a sensor that is configured to measure impedance at two or more frequencies of two or more sensing regions in operational contact with a fluid containing one or more analytes. The sensor may include a plurality of sensing elements corresponding to a plurality of sensing regions that are configured to sense the analyte. The sensor may also include a controller coupled to the plurality of sensing regions, wherein the controller is configured to receive an indication of the sensed analyte. The sensor system may also include an impedance measurement system that is coupled to the controller to receive the indication from the controller. The impedance measurement system may include a plurality of processors, where a first processor of the plurality of processors is configured to provide, based on the sensed analyte, one or more re-configurable frequency measurements over a frequency range.

In another embodiment, an impedance measurement system includes a plurality of sensing elements configured to sense one or more analytes, where each sensing element of the plurality of sensing elements corresponds to a particular sensing region of a plurality of sensing regions, and where each sensing region of the plurality of sensing regions is in operational contact with a fluid containing the one or more analytes. The impedance measurement system may also include one or more processors configured to measure one or more impedance responses of two or more sensing regions of the plurality of sensing regions concurrently at two or more frequencies or concurrently from two or more sensing regions of the plurality of sensing regions. The impedance measurement system may also include control circuitry communicatively coupled to the plurality of sensing regions and the one or more processors.

In another embodiment, an impedance measurement system chip assembly may include sensing materials that are applied onto individual sensing regions, where each region of the plurality of sensing regions is configured to sense a gas, chemical, physical, or biological analyte disposed in a fluid. The impedance analyzer chip assembly may also include a plurality of processors. A first processor may be configured to output one or more re-configurable impedance values across a frequency range of at least six orders of magnitude. A second processor may be configured to output one or more re-configurable impedance values from one or more of the sensing regions of the plurality of sensing regions. A third processor may be configured to output two or more impedance values from two or more of the sensing regions of the plurality of sensing regions. A fourth processor may be configured to concurrently output one or more impedance values at two or more frequencies. A fifth processor may be configured to perform on-board data analytics from responses of two or more sensing regions of the plurality of sensing regions to the fluid containing the gas, chemical, physical, or biological analyte. The chip assembly may also include an exterior housing in which the plurality of sensing regions and the plurality of processors are disposed.

### BRIEF DESCRIPTION OF THE DRAWINGS

FIGS. 1A, 1B, and 1C illustrate non-limiting examples of various use contexts for a multi-parameter sensor system, in accordance with one or more embodiments;

FIG. 2 illustrates a diagram of the multi-parameter sensor system embodiment, in accordance with one embodiment; and

FIG. 3 illustrates a diagram of a multi-parameter impedance measurement system in a chip assembly embodiment, in accordance with one embodiment.

### DETAILED DESCRIPTION

One or more embodiments of the subject matter described herein provide systems and methods that provide a multi-channel impedance measurement system in an integrated chip assembly for multi-parameter sensing of at least one analyte. The systems and methods may allow for detection of gas, chemical, physical, and/or biological analyte species (e.g. liquids or gases) in various contexts and applications with improved sensitivity and accuracy.

Complex environments, such as industrial, urban, medical, or military, may contain many different analytes (e.g. liquids and/or gases) to be detected and measured. Some sensors may provide continuous monitoring capabilities yet have poor detection selectivity of a particular analyte over others. Additionally, some sensors may be designed to provide a single output per sensor. Sensors with a single output per sensor are known as conventional sensors or zero-order sensors. Therefore, in order to sense multiple different analytes in a variety of contexts or environments, multiple different conventional sensors may be needed, which may be costly and inconvenient. Further, having multiple different conventional sensors may be difficult to calibrate and preserve the calibration over time upon exposure to the complex environments, as each conventional sensor in the system may require its own calibration function stored onboard the monitor. Additionally, any conventional or zero-order sensor is undesirably affected by variable chemical background and sensor drift that cannot be distinguished from the response to an analyte.

Additionally, some such conventional sensors or analytical instruments may utilize analytical technologies that are costly, large, have a narrow dynamic range of sensing, and require relatively high-power consumption (e.g. chromatography, mass spectrometry, ion mobility spectrometry, tunable diode laser absorption spectroscopy). Even some wearable personal multi-gas monitors or systems may require or include multiple different types of conventional sensors for detecting of different species of analytes, which may require multiple sensor elements and, therefore, lead to an increase in power consumption and need-dedicated electronics for each of the sensors. As such there is a need for more convenient and less-costly sensor system that may be used to detect and measure a variety of analyte species in a variety of use contexts.

Disclosed herein is a transportable and a microelectronic sensor system capable of detecting and measuring multiple species of analytes in various use contexts. The multi-channel integrated systems and methods for multi-parameter gas, biological, chemical, and physical sensing described herein utilizes multi-variable detection that may be performed using multi-frequency impedance measurements. The impedance measurement system may be implemented in a sensor system. The impedance measurement system may include a multi-variable sensor or also known as multi-parameter sensor that may be configured to provide independent outputs and to recognize different responses from the measured environment. Multi-variable sensors with multiple outputs are also known as the first-order sensors. The first-order sensors address the problems of zero-order sensors. Sensor measurements may be performed using integrated circuit impedance analyzers. The sensor system may also include a multi-core processor configured to: (1) provide re-configurable frequency range of measurements (e.g., over one, two, three, four, five, six, or another order of magnitude of frequency range); (2) provide a re-configurable readout of impedance values from a single sensing region and/or multiple sensing regions; (3) simultaneously readout impedance values from multiple sensing regions; and (4) simultaneously readout impedance at multiple frequencies.

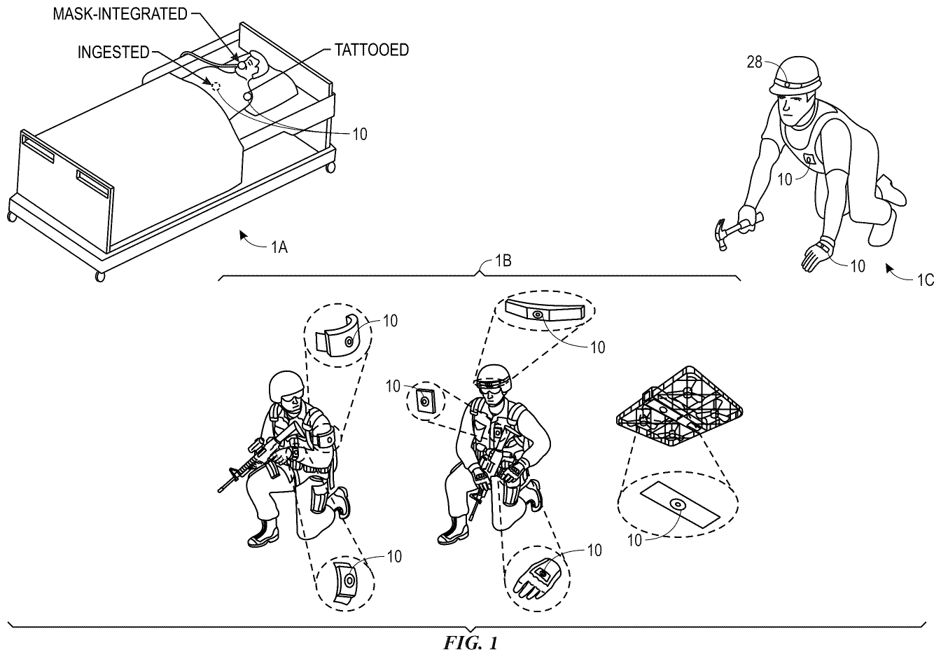

With the foregoing in mind, FIG. 1 illustrates non-limiting examples of various use contexts for a multi-parameter sensor system, in accordance with one or more embodiments. In particular, FIGS. 1A, 1B, and 1C illustrate some non-limiting examples of these various embodiments of how the multi-parameter sensor system 10 may be used and the diverse contexts it may be applied in, such as patient monitoring (FIG. 1A), military monitoring (FIG. 1B), or industrial monitoring (FIG. 1C). In some embodiments, the sensor system 10 may be a portable, transportable, ingestible, implantable, or wearable device that may be worn, carried, ingested, implanted, and/or otherwise easily moved from one place to another by an operator. Further, the sensor system 10 may be an indoor device and/or an outdoor device. In some embodiments, the sensor system 10 may be an industrial environmental sensor, an asset monitoring sensor, a safety sensor, an engine sensor, a gas turbine sensor, an industrial process monitoring gas sensor, a personal consumer sensor, a transportation sensor, a security sensor, or any combination thereof. In some embodiments, the sensor may be part of a wireless sensor network.

For example, the sensor 10 may be positioned in or be an integrated part of a helmet, hat, glove, or other clothing attributes. In another example, the sensor 10 may be held within a wearable or non-wearable transferable object, such as a frame of military or industrial eyeglasses, a wearable pulse oximeter, a safety vest or harness, an article of clothing, a mobile device (e.g., a cellular phone, a tablet, or the like), or the like. In another wearable device embodiment, the sensor 10 may be integrated into a fabric of the clothing or positioned on clothing such as on a pocket. The sensor 10 may be in a form of an arm band, worn on a wrist or other extremity, or the like. In another wearable device embodiment, the sensor 10 may be worn by any subject including a human or a plant or an animal or a robot. In some embodiments, the sensor 10 may be removably coupled or integrated with an article worn by a subject (e.g., a shirt, pants, safety vest, safety personal protection clothing, eyeglasses, hat, helmet, hearing device, or the like). In some embodiments, the sensor 10 may be any alternative device that is portable or that may be transferrable such that sensor 10 may be moved between different positions. Additionally, or alternatively, the sensor 10 may be stationary or substantially stationary. In another wearable device embodiment, the sensor 10 may be coupled with, integrated with, disposed on, or the like, an asset, such as a moving system such as a drone, a stationary system, or the like. Further, in this way, the sensor 10 may be worn, or otherwise carried, by different subjects or individuals, such as, but not limited to, soldiers, medical professionals, athletes, system operators, students, otherwise active or inactive individuals, or the like. It should be noted that, regardless of the application or context, in some embodiments, the sensor system 10 may be applied for single-use such that it is intended to be discarded after the first use. Additionally, or alternatively, the sensor system 10 may be applied for temporary (e.g. three to five uses, use based on an amount of time, or use for specific purpose) or long-term use.

In such embodiments as those described with respect to FIGS. 1A, 1B, and 1C, any device comprising the sensor system 10 may be fabricated using manufacturing technologies based on complementary metal-oxide-semiconductor electronics, flexible electronics, flexible hybrid electronics and other known approaches to provide conformal and flexible designs, implementations, and use. Further, it should be noted that FIG. 1 depicts only a few implementations of the sensor 10 and is not exhaustive.

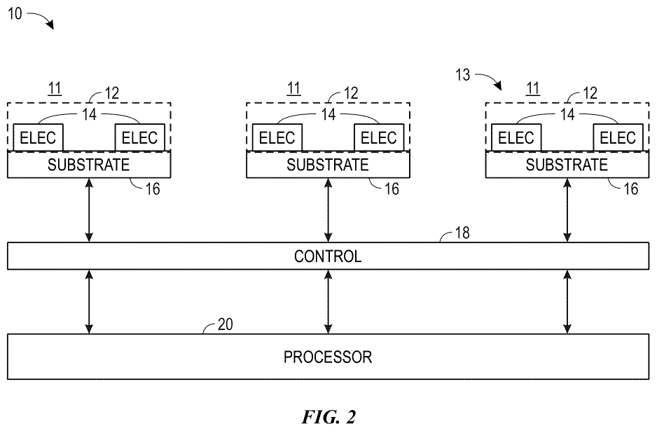

FIG. 2 provides a diagram of an embodiment of the multi-parameter sensor system for multi-variable analysis of response of the sensor to one or more analytes in a fluid sample, in accordance with the present technique. The fluid may be a gas, a liquid, a gas-liquid mixture, particles or particulate matter, or the like, containing one or more analytes therein. By way of example, in one implementation the fluid may be indoor or outdoor ambient air. Another example of the fluid is air at an industrial site, residential site, military site, battlefield site, construction site, urban site, or any other known site. Still, another example of the fluid may be transformer oil or any insulating fluid of an electrical equipment that is installed and/or disposed of below a ground level, above the ground level, near to the ground level, or any other position. In another embodiment, the fluid may be a gas or fuel, such as a hydrocarbon-based fuel or a hydrogen fuel. One example of the fluid is natural gas that is supplied to a powered system (e.g., a vehicle, or a stationary generator set) for consumption. Another example of the fluid is hydrogen gas that is supplied to a powered system (e.g., a road vehicle, an aircraft engine, or a stationary generator set) for consumption. Other examples of such a fluid can include, but are not limited to, gasoline, diesel fuel, jet fuel or kerosene, bio-fuels, petrodiesel-biodiesel fuel blends, natural gas (liquid or compressed), and fuel oils. Another example of the fluid is at least one gas dissolved in a consumer liquid such as milk, non-alcoholic beverages, alcoholic beverages, cosmetics, and so forth. Another example of the fluid is at least one analyte dissolved in a bodily liquid such as blood, sweat, tears, saliva, urine, and so forth. Another example of the fluid is ambient air with relatively small concentrations of benzene, naphthalene, carbon monoxide, ozone, formaldehyde, nitrogen dioxide, sulfur dioxide, ammonia, hydrofluoric acid, hydrochloric acid, phosphine, ethylene oxide, carbon dioxide, hydrogen sulfide, chemical warfare agents such as nerve, blister, blood, and choking agents, hydrocarbons and/or other pollutants. Another example of the fluid is ambient air with relatively small concentrations, medium concentrations, and large concentrations of flammable or combustible gases such as methane, ethane, propane, butane, hydrogen, and/or other gases. Another example of the fluid is SF6 gas and its decomposition products. Another example of the fluid is g3 gas mixture and its decomposition products. Another example of the fluid is gas or liquid with analyte particles in the fluid where the analyte particles are inorganic particles, polymeric particles, or/and biological particles. Non-limiting examples of inorganic particles include urban and/or rural dust particles, coal dust particles, forest fire and any other fire particles. Non-limiting examples of polymeric particles or moieties include per- and poly-fluoroalkyl substances (PFASs). Non-limiting examples of biological particles include viruses, bacteria, fungi, allergens and others.

The sensor system 10 may include at least two sensors 13 corresponding to two or more sensing regions 11 and having same or different sensing materials 12, two or more electrodes 14, and two or more substrates 16. The system 10 may also include a controller 18 (e.g. control circuitry) and an impedance measurement system 20. The system 10, via the aforementioned components, may be configured to measure impedance at two or more frequencies of the two or more sensing regions in contact with the fluid containing the analyte.

The sensing materials 12 may be a metal-oxide semiconductor (MOS) sensing materials or it may be non-MOS sensing materials 12 such as a dielectric polymer material, conducting polymer material, bio-receptor material, nanotubes material, nanowires material, nanoparticles material, metal organic frameworks material, graphene material, supramolecular compound material, a MXene which is a two-dimensional inorganic material consisting of atomically thin layers of transition metal carbides, nitrides, or carbonitrides, and/or other materials. The sensing materials 12 may be applied onto two or more sensing regions 11 that operate to sense an analyte via, for example, dielectric excitation methods, as explained in more detail below. The sensors 13 and the corresponding sensing regions 11 may include two or more electrodes 14 in contact with a substrate 16. As shown in FIG. 2, the electrodes 14 may be disposed on the substrate 16. The sensing regions 11, along with the electrodes 14 and substrate 16, may be in contact with a fluid sample containing the analyte to be detected.

In some embodiments, excitation or dielectric excitation of the sensing materials 12 and measurement of excitation responses of the sensing materials 12 may be performed via electrodes 14. The sensing materials 12 may be able to interact with at least one analyte and may be exposed to the sample fluid containing at least one analyte. The sensing regions 11 having two or more electrodes electrically coupled to the control circuitry or controller 18 and the impedance measurement system 20 may include sensing materials 12. The controller 18 may include viable power supply and excitation circuitry configured to provide one or more excitation signals to the electrodes 14, such as an alternating current at least one predetermined frequency or predetermined frequency range. That is, in some embodiments, the sensor system 10 may apply dielectric excitation (e.g. an alternating current (AC)) to the sensing materials 12 at multiple excitation frequencies. The power supply may be a stand-alone electrical power supply or other circuitry configured to couple to or integrate with an asset to share electrical power with the asset.

The impedance measurement system 20 may include viable measurement circuitry configured to receive and measure one or more excitation responses of the electrodes 14 to the electrical signal with the alternating current at the at least one predetermined frequency. The impedance measurement system 20 may be coupled to the two or more sensing regions 11 and may be able to receive an indication of the sensed analyte from the two or more sensing regions 11. The impedance measurement system 20 may measure the induced changes on the sensing materials 12 and/or the sensing regions 11 upon receiving the excitation responses. The excitation responses may indicate one or more characteristics of induced changes on the sensing materials 12 and/or the sensing region 11 as a function of analyte composition and/or concentration within the fluid.

For example, the impedance measurement system 20 may measure impedance, real part of impedance (e.g. Z′, Zre), imaginary part of impedance (e.g. Z″, Zim), admittance, reactance, susceptance, capacitance, electrical current, or a combination thereof, among other things of the excitation responses. As used herein, the term “impedance” may be a non-limiting term for any electrical response to an electrical current applied to the system resulting in induced changes on the excitation signals when measuring the excitation responses. In some embodiments, impedance measurements may be performed at a single frequency, at discrete frequencies, or at multiple scanned frequencies. Further, in some embodiments, impedance measurements may be performed by one or more impedance analyzers or impedance analyzer circuits that may be a part of or coupled with the impedance measurement system 20.

In some embodiments, the impedance measurement system 20 may apply a transfer function, a multiplier coefficient, a lookup table, a model, among other things, to data collected from the impedance response and/or the DC response of the sensing materials 12 and/or the two or more sensing regions 11 to identify one or more gas, chemical, physical, or biological species and/or concentration of such species that may be present in the analyte fluid sample. To accomplish this, each sensing region 11 may be configured for sensing gas, chemical, physical, or biological species. The sensing regions 11 may be coupled to the controller 18, the impedance measurement system 20, or both. Additionally or alternatively, one or more sensing regions of the two or more sensing regions 11 may be configured to serve as a reference sensing region to provide a reference value.

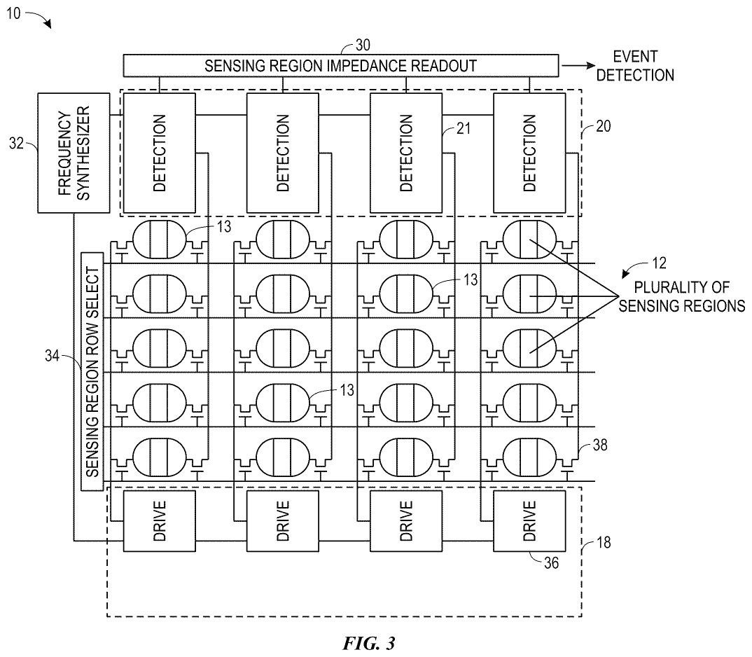

For example, as explained in more detail below with respect to FIG. 3, the impedance measurement system 20 may include or be coupled to components and circuitry (e.g. two or more sensing regions 11) that are configured to detect multiple species of the analyte disposed in the fluid sample by performing measurements at AC and/or DC measurement conditions and/or impedance measurement conditions. The impedance measurement system 20 may also be coupled to the controller 18 and, therefore, receive the indication of excitement from the controller 18. Accordingly, the controller 18 and/or the impedance measurement system 20 may determine chemical, physical, and biological analyte species (and/or concentration of such species) present in the fluid sample based on the measured responses of the at least two or more sensing regions 11 by the impedance measurement system 20. In some embodiments, this may be achieved by the controller 18 via on-board data processing and analytics of the measured dielectric excitation responses at each sensing region 11.

Further, the impedance measurement system 20 may include a processor comprising one or more impedance measurement components configured to perform multi-parameter sensing. In another embodiment, the impedance measurement system 20 may be a multi-core processor comprising two or more processors. Each processor of the multi-core processor may include impedance measurement components configured to perform multi-parameter sensing. In this way, the multi-parameter sensor system 10 may detect chemical, physical, and biological species in application-specific contexts.

FIG. 3 is a diagram of an example embodiment of the multi-parameter impedance measurement system in a chip assembly. That is, the multi-parameter impedance measurement system may be implemented as an application-specific integrated circuit (ASIC) impedance analyzer with sensing elements coupled to the ASIC. FIG. 3 demonstrates a schematic, non-limiting example of the electrical components and arrangement of the impedance measurement system 20 with sensing elements and individually addressable sensing regions 11. In some embodiments, the impedance measurement system 20 may be monolithically integrated with two or more sensing regions 11 for gas, chemical, physical, and biological sensing.

The chip assembly embodiment of the multi-parameter impedance measurement system 10 may include similar elements to those described in FIG. 2. For example, the chip assembly 10 may include sensing materials 12, two or more sensing regions 11, a controller 18, and the impedance measurement system 20. The controller 18 may include one or more drives 36 that may operate to provide excitation methodologies applied to the two or more sensing regions 11 in response to one or more instructions from the controller 18. The one or more drives may include one or more mechanisms for providing the necessary excitation or operating conditions (e.g. electric excitation) for detection of an analyte. For example, in some embodiments, the one or more drives 36 may include a voltage and/or current source.

The one or more drives 36 may be coupled to a corresponding row of sensing regions 38 of a plurality of sensing regions 11 and/or a corresponding processor 21 of a plurality of processors of the impedance measurement system 20. The one or more drives 36 may be configured to provide excitation (e.g. electrical pulses) to the sensing regions 11. In some embodiments, the drives 36 may provide such excitation based on input via the controller 18, which may include information such as the applied context, a species of analyte, or other information regarding the specific use or sensing context. The input may be operator input, input from a result of on-board data analytics, an input from a result of off-board analytics, an input from a result of remote server data analytics, or any combination thereof. The input may signal the controller 18 how to operate and, therefore, determine which sensing regions 38 or row of sensing regions 38 is being excited or stimulated. In this way, an operator may program the controller 18 to operate in a particular fashion depending on context. Further, in this way, the controller 18, based on the input, may be able to control which sensing region of the plurality of sensing regions 11 will provide the re-configurable readout of impedance values based on an input and/or instruct the impedance measurement system 20 as to which sensing region of the sensing regions 11 will provide the readout. Once stimulated, the impedance measurement system 20 may detect a response of the sensing regions 11.

The impedance measurement system 20 may include a processor or a plurality of processors 21. Further, the impedance measurement system 20 may include or be implanted in an integrated circuit, a monolithically integrated circuit, or the like. In particular, when applying excitation methodologies similar to those described above, the impedance measurement system 20 may include one or more processors 21 (e.g. a single processor or a multi-core processor) that are configured to measure impedance responses (e.g., Z′ and Z″) of the two or more sensing regions 11 with re-configurable frequency ranges. In this way, the impedance measurement system 20 may provide capability to measure sensor impedance responses at multiple discrete frequencies and/or across a frequency range. As described above, the term “impedance” as used herein may be a non-limiting term for any electrical response to an electrical current applied to the system resulting in induced changes on the excitation signals when measuring the excitation responses. The excitation responses may indicate one or more characteristics of induced changes on the sensing materials 12 and/or the sensing region 11 as a function of analyte composition and/or concentration within the fluid. The one or more processors 21 may also be configured to measure impedance responses (e.g., Z′ and Z″) of the two or more sensing regions 11 with re-configurable addresses and number of sensing regions. For example, the one or more processors may re-configure which sensing regions 11 as well as how many sensing regions 11 to measure the impedance responses of. This may be done via the controller 18 and/or row-select circuitry 34 that operates to selectively transmit signals to activate rows of sensing regions 11 based on input or other predetermined operation parameters. For example, the controller 18 may signal the row-select circuitry 34 what row of sensing regions 38 to activate. In response to the signal, the row-select circuitry 34 may operate a set of switches that send a signal to active the indicated row. The one or more processors 21 may also be configured to measure impedance responses (e.g., Z′ and Z″) of the two or more sensing regions 11 with simultaneous or concurrent readout from multiple sensing regions 11. The one or more processors 21 may also be configured to measure impedance responses (e.g., Z′ and Z″) of the two or more sensing regions 11 simultaneously or concurrently at multiple frequencies.

In some embodiments, the impedance measurement system 20 may include a processor configured to re-configure a frequency range of measurements (e.g., over one, two, three, four, five, six, or another order of magnitude of frequency range). The impedance measurement system 20 may include a processor (or processors) configured to provide a re-configurable readout of impedance values from a two sensing region to more sensing regions. In some embodiments, the impedance measurement system 20 may include a processor configured to provide simultaneous readout of impedance values from multiple sensing regions. In some embodiments, the impedance measurement system 20 may include a processor configured to provide simultaneous readout of impedance at multiple frequencies.

In certain embodiments, the impedance measurement system 20 may also include one or more output devices, such as a sensing region impedance readout device 30, configured to output the detected event. The readout device 30 may include a port, an output or a display configured to present information regarding the detected event (e.g. impedance event/impedance analysis). For example, the readout device 30 may indicate (e.g. via the display) a classification and/or concentration of two or more analyte species in the fluid sample. In alternative or additional embodiments, the readout devices 30 may include an alarm, such as visual alarms (e.g., light emitting diodes (LEDs)), auditory alarms (e.g., speakers), and/or haptic alarms (e.g., haptic feedback devices). In this way, the impedance measurement system 20 may be configured to perform and provide on-board data analysis from responses of two or more sensing regions of the plurality of sensing regions 11 to the fluid containing the one or more analytes. Alternatively or additionally, the sensing region impedance readout device 30 may include one or more communication devices (e.g., wired communication interfaces, wireless communication interfaces) that may enable the chip assembly 10 to communicate with other computing systems, such as a desktop computer, a mobile computing device (e.g., a laptop, smart phone), a remote server (e.g., an Internet server, a cloud server), or other sensors (e.g., gas sensors, temperature sensors, vibration sensors, health monitors) of a multi-sensor monitoring system. For example, in some embodiments, information determined by the one or more processors of the impedance measurement system 20 may be provided to an external computing system that serves as a controller of a mesh of sensors that includes a sensor implementing the chip assembly 10. In some embodiments, the sensing region impedance readout device 30 may additionally or alternatively use the communication devices to provide excitation response measurements to an external computing system, such that the external computing system may use these measurements to calculate one or more coefficient values for one or more of the gas analysis models. In certain embodiments, the impedance measurement system 20 may also include a frequency synthesizer 32. The synthesizer 32 may include an oscillator to generate one or more frequencies of excitation.

As used herein, an element or step recited in the singular and proceeded with the word “a” or “an” should be understood as not excluding plural of said elements or steps, unless such exclusion is explicitly stated. Furthermore, references to “one embodiment” of the presently described inventive subject matter are not intended to be interpreted as excluding the existence of additional embodiments that also incorporate the recited features. Moreover, unless explicitly stated to the contrary, embodiments “comprising,” “including,” or “having” (or like terms) an element, which has a particular property or a plurality of elements with a particular property, may include additional such elements that do not have the particular property.

As used herein, terms such as “system” or “controller” may include hardware and/or software that operate(s) to perform one or more functions. For example, a system or controller may include a computer processor or other logic-based device that performs operations based on instructions stored on a tangible and non-transitory computer readable storage medium, such as a computer memory. Alternatively, a system or controller may include a hard-wired device that performs operations based on hard-wired logic of the device. The systems and controllers shown in the figures may represent the hardware that operates based on software or hardwired instructions, the software that directs hardware to perform the operations, or a combination thereof.

As used herein, terms such as “operably connected,” “operatively connected,” “operably coupled,” “operatively coupled,” “operationally contacted,” “operational contact” and the like indicate that two or more components are connected in a manner that enables or allows at least one of the components to carry out a designated function. For example, when two or more components are operably connected, one or more connections (electrical and/or wireless connections) may exist that allow the components to communicate with each other, that allow one component to control another component, that allow each component to control the other component, and/or that enable at least one of the components to operate in a designated manner.

As used herein, terms such as “concurrently,” “simultaneously,” and the like indicate that two or more objects or events are existing, occurring, operating, happening, or are done at the same time. Such terms may be used herein interchangeably.

It is to be understood that the subject matter described herein is not limited in its application to the details of construction and the arrangement of elements set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the inventive subject matter, and also to enable one of ordinary skill in the art to practice the embodiments of inventive subject matter, including making and using any devices or systems and performing any incorporated methods. The patentable scope of the inventive subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A sensor system, comprising:
a sensor configured to measure impedance at two or more frequencies of two or more sensing regions in operational contact with a fluid containing one or more analytes, wherein the sensor comprises:
a plurality of sensing elements corresponding to the two or more sensing regions configured to sense the one or more analytes; and
a controller coupled to the two or more sensing regions, wherein the controller is configured to receive an indication of the sensed one or more analytes; and
an impedance measurement system coupled to the controller to receive the indication from the controller, wherein the impedance measurement system comprises a plurality of processors, wherein a first processor of the plurality of processors is configured to provide, based on the sensed one or more analytes, one or more re-configurable frequency measurements over a frequency range.

2. The sensor system of claim 1, wherein the first processor is configured to provide, based on the sensed one or more analytes, the one or more re-configurable frequency measurements over a frequency range of at least six orders of magnitude of frequency range.

3. The sensor system of claim 1, wherein each analyte of the one or more analytes is a chemical analyte, a physical analyte, a biological analyte, or any combination thereof.

4. The sensor system of claim 1, wherein at least a portion of the impedance measurement system is housed within a common enclosure with the sensor.

5. The sensor system of claim 1, wherein the impedance measurement system comprises a second processor of the plurality of processors configured to provide a re-configurable readout of impedance values based on the sensed one or more analytes from two or more sensing regions of the plurality of sensing regions.

6. The sensor system of claim 5, wherein the controller is configured to control which sensing regions of the plurality of sensing regions will provide the re-configurable readout of impedance values based on an input.

7. The sensor system of claim 6, wherein the input is an operator input, an input from a result of on-board data analytics, an input from a result of off-board analytics, or an input from a result of a remote server data analytics.

8. The sensor system of claim 1, wherein a second processor of the plurality of processors is configured to provide on-board data analysis from responses of two or more sensing regions of the plurality of sensing regions to the fluid containing the one or more analytes, wherein the on-board data analysis is based on one or more responses of the sensor at two or more measured frequencies over a frequency range.

9. The sensor system of claim 1, where one or more sensing regions of the two or more sensing regions are configured to serve as a reference sensing region to provide a reference value.

10. The sensor system of claim 1, wherein a third processor of the plurality of processors is configured to concurrently readout impedance values from multiple sensing regions of the plurality of sensing regions.

11. The sensor system of claim 1, wherein the impedance measurement system comprises an integrated circuit.

12. The sensor system of claim 1, wherein the sensor system comprises a disposable, single-use system or a wearable device worn by an operator, an implantable device, an ingestible device, an outdoor device, an indoor device, a mobile device, a stationary device, or any combination thereof.

13. The sensor system of claim 1, wherein the impedance measurement system comprises a monolithically integrated system comprising a single chip configured to monitor all sensing regions of the plurality of sensing regions.

14. The sensor system of claim 1, wherein the sensor system is configured to be powered via a stand-alone power or shared power when integrated into an asset.

15. An impedance measurement system, comprising:
a plurality of sensing elements configured to sense one or more analytes, wherein each sensing element of the plurality of sensing elements corresponds to a sensing region of a plurality of sensing regions, and wherein each sensing region of the plurality of sensing regions is in operational contact with a fluid containing the one or more analytes;
an impedance analyzer comprising one or more processors configured to measure one or more impedance responses of two or more sensing regions of the plurality of sensing regions concurrently at two or more frequencies or concurrently from two or more sensing regions of the plurality of sensing regions; and
control circuitry communicatively coupled to the plurality of sensing regions and the one or more processors.

16. The impedance measurement system of claim 15, comprising an application-specific integrated circuit (ASIC).

17. The impedance measurement system of claim 16, wherein the ASIC is disposed in a single chip and is configured to monitor all sensing regions of the plurality of sensing regions.

18. The impedance measurement system of claim 15, wherein the one or more processors are configured to output, based on the sensed one or more analytes, one or more re-configurable frequency measurements over a frequency range.

19. The impedance measurement system of claim 15, wherein the one or more processors are configured to output, based on the sensed one or more analytes, one or more re-configurable measurements over a frequency range of at least six orders of magnitude.

20. An impedance measurement system chip assembly, comprising:
sensing materials deposited onto a plurality of sensing regions, wherein each region of the plurality of sensing regions is configured to sense a gas, chemical, physical, or biological analyte disposed in a fluid;
a plurality of processors comprising:
a first processor configured to output one or more re-configurable impedance values across a frequency range of at least six orders of magnitude;
a second processor configured to output one or more re-configurable impedance values from one or more of the sensing regions of the plurality of sensing regions;
a third processor configured to concurrently output two or more impedance values from two or more of the sensing regions of the plurality of sensing regions;
a fourth processor configured to concurrently output one or more impedance values at two or more frequencies; and
a fifth processor configured to perform on-board data analytics from responses of two or more sensing regions of the plurality of sensing regions to the fluid containing the gas, chemical, physical, or biological analyte, wherein the data analytics is based on one or more responses of the two or more sensing regions at two or more measured frequencies over a frequency range; and
an exterior housing in which the plurality of sensing regions and the plurality of processors are disposed.
